(19) United States
(12) Patent Application Publication
(10) Pub. No.: US 2022/0387303 A1
(43) Pub. Date: Dec. 8, 2022

(54) Vaginal Drug Delivery Device

(71) Applicant: THE REGENTS OF THE UNIVERSITY OF CALIFORNIA, Oakland, CA (US)

(72) Inventors: Leena Nathan, Oakland, CA (US); Tamara Grisales, Oakland, CA (US); Song Li, Oakland, CA (US); Mohammad Mahdi Hasani Sadrabadi, Oakland, CA (US)

(21) Appl. No.: 17/771,320

(22) PCT Filed: Oct. 22, 2020
(86) PCT No.: PCT/US20/56928

§ 371 Date: Apr. 22, 2022

Related U.S. Application Data
(60) Provisional application No. 62/924,376, filed on Oct. 22, 2019.

(51) Int. Cl. A61K 9/00; A61M 37/00

(52) U.S. Cl. CPC A61K 9/0021; A61M 37/0015; A61M 2037/0053; A61M 2037/0023

(57) ABSTRACT

The present invention relates to a vaginal drug delivery device configured to safely and effectively deliver a therapeutic formulation in the vagina at or near a desired target area for a specified time period.

### CROSS-REFERENCE TO RELATED APPLICATIONS

This application claims priority to U.S. Provisional Patent Application No. 62/924,376, filed Oct. 22, 2019, the contents of which are incorporated by reference herein in its entirety.

### BACKGROUND OF THE INVENTION

Vaginal epithelial tissue has been shown to be receptive to drugs delivered by many different therapeutic formulations. However, it is difficult to maintain therapeutic agents in place on vaginal surfaces and replenish depleted areas with a fresh agent. Vaginal gels, foaming tablets, and creams are messy in application and prone to leakage. This problem is further complicated by the fact that an ideal location for topical therapeutic vaginal drug delivery is commonly at or near the woman's cervix.

Thus, there exists a need for a convenient method that a woman can use that is safe and effective in keeping a delivered therapeutic formulation in the vagina at or near a desired target area for a specified/sufficient time period. The present invention meets this need.

### SUMMARY OF THE INVENTION

In one aspect the present invention provides a vaginal drug delivery device comprising: a flexible base; and a plurality of microstructures protruding from the base, wherein the microstructures each comprise a proximal end, a distal end, a body and a tip. In one embodiment, the microstructures are selected from the group consisting of microneedles, microblades, microanchors, microfishscale, micropillars, microhairs and combinations thereof. In one embodiment, the microstructures each comprises a length ranging from about 250-1000 μm. In one embodiment, the microstructures each comprises a cross sectional diameter ranging from about 80-600 μm at the proximal end. In one embodiment, the base is biodegradable. In one embodiment, the plurality of microstructures are biodegradable. In one embodiment, the base has a shape selected from the group consisting of: a tablet, a film, a ring, a capsule, and combinations thereof. In one embodiment, the device is made from polymers selected from the group consisting of: Polyesters comprising Poly(glycolic acid), Poly(lactic acid), Poly(lactic-glycolic acid) and Poly(caprolactone) (PCL); Polysaccharides comprising chitosan, dextran, alginate, hyaluronic acid; Polyanhydrides; Polyorthoesters; Polyurethanes and combinations thereof. In one embodiment, the plurality of microstructures further comprise a therapeutic agent. In one embodiment, the therapeutic agent is a drug. In one embodiment, the drug is selected from the group consisting of: estrogen, progesterone, estradiol, an antibacterial agent, an antifungal agent, and combinations thereof. In one embodiment, the device has a degradation rate of about 4 days to 9 months after insertion. In one embodiment, the device has a drug release rate of about 5-20 μg per day. In one embodiment, the body is curved along its length between the proximal end and the distal end. In one embodiment, the body connects the proximal end and the distal end without any curvature along its length. In one embodiment, the tip is selected from the group consisting of: a cube, a rectangle, a sphere, a cone, a pyramid, a cylinder, a tube, a ring, a tetrahedron, a hexagon, an octagon, or any irregular shapes.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts an exemplary vaginal delivery device of the present invention.

FIG. 2 comprising FIG. 2A through FIG. 2D depicts various exemplary forms of the vaginal drug delivery device of the present invention. FIG. 2A depicts an exemplary vaginal drug delivery device of the present invention in a tablet form. FIG. 2B depicts an exemplary vaginal drug delivery device of the present invention in a film form. FIG. 2C depicts an exemplary vaginal drug delivery device of the present invention in a ring form. FIG. 2D depicts an exemplary vaginal drug delivery device of the present invention in a capsule form.

FIG. 3 comprising FIG. 3A through FIG. 3B depicts the fabrication process of drug delivery device. FIG. 3A depicts melt mixing as an industrial standard process to form drug delivery devices. Here polymer pellets were molten and mixed with the drug using micro-extruders prior to casting and hot pressing into the mold to fabricate the device with target geometry. FIG. 3B depicts solvent mixing and membrane emulsification technique that were used to form sub-millimeter size drug-polymer microparticles. These particles were washed, dried and then casted into the molds prior to hot pressing to fabricate the device with target geometry.

FIG. 4 depicts cumulative release estradiol from PCL device for 70 days at 37° C. under gentle shaking. Data presented as average ±SD. The average drug dose per day were also calculated based on the release data.

FIG. 5 depicts estrogen receptor responsive luciferase reporter t47d stable cell line is used to assess change in the activity of estradiol after being released from PCL device at different timepoints up to 7 weeks. (RLU: relative luminescence unit). Data presented as average ±SD.

FIG. 6 depicts cumulative release progesterone from PCL device for 70 days at 37° C. under gentle shaking. Data presented as average ±SD. The average drug dose per day were also calculated based on the release data.

FIG. 7 depicts cumulative release progesterone from PCL device for 70 days at 37° C. under gentle shaking. Data presented as average ±SD. The average drug dose per day were also calculated based on the release data.

FIG. 8 depicts cumulative release profiles of Clotrimazole and Metronidazole progesterone from PCL device for 70 days at 37° C. under gentle shaking. Data presented as average ±SD. The average drug dose per day were also calculated based on the release data.

FIG. 9 depicts antibacterial properties of antibacterial delivery devices were measured by estimating the inhibition zone from the disk diffusion assay.

FIG. 10 depicts in vivo biocompatibility of devices. Surgical implantation of device subcutaneously in wild-type mice were used to test the local and systematic toxicity of PCL and PLGA-based devices one week after insertion. Whole-blood analysis of mice after implantation with PCL or PLGA devices. white blood cells, red blood cells, and platelets counts were shown. Comprehensive metabolic screening of mice after implantation with various devices. Liver function assessment: ALT (alanine aminotransferase), AST (aspartate aminotransferase), BUN (blood urea nitrogen), LDH (Lactate dehydrogenase). Kidney function assessment: Creatinine and total protein content were measured and compared between groups.

### DETAILED DESCRIPTION

It is to be understood that the figures and descriptions of the present invention have been simplified to illustrate elements that are relevant for a clear understanding of the present invention, while eliminating, for the purpose of clarity, many other elements found in the relevant field. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Vaginal Drug Delivery Device

The present invention relates to a drug delivery device for delivery of therapeutic formulations to or through vaginal surfaces. In one embodiment, the vaginal delivery device is able to treat gynecologic conditions that require the use of vaginal medication including but not limited to infertility, vaginal infections and genitourinary syndrome of menopause/vaginal atrophy such as vaginal dryness/pain, recurrent urinary tract infections (UTIs), overactive bladder and urinary incontinence. In one embodiment, the vaginal medications used with the delivery device comprise but are not limited to estrogen, progesterone, estradiol, and suitable antibacterial and antifungal agents.

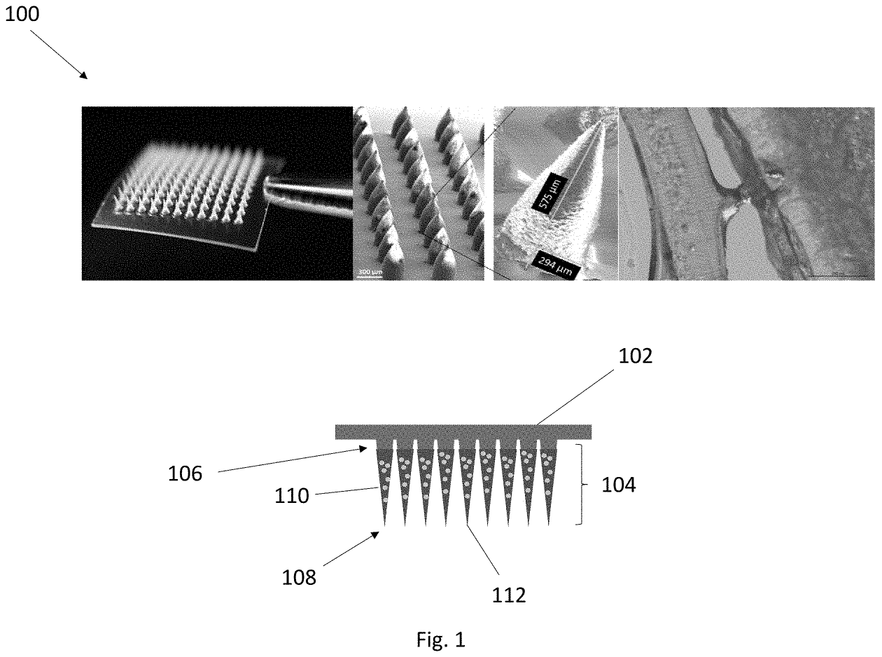

Referring to FIG. 1, an exemplary vaginal delivery device of the present invention is shown. Vaginal delivery device 100 comprises a base 102 and a plurality of microstructures 104 protruding from base 102.

Base 102 can be made of a stretchable and breathable material. Alternatively, Base 102 can be made of any suitable material. In some embodiments, for example, base 102 can be made of a material that is transparent, or substantially transparent. In other embodiments, base 102 can be made of a material that is not transparent. In one embodiment, base 102 may be made from natural, synthetic, and/or artificial materials; and in some particular embodiments, they comprise a polymeric substance. Base 102 may be comprised of materials that are nontoxic, biodegradable, bioresorbable, or biocompatible.

Alternatively, the flexibility and/or stretchability of base 102 may vary across, or along, vaginal drug delivery device 100. Further, in some embodiments, base 102 can comprise elastic properties, wherein the elasticity may optionally be similar throughout base 102. Alternatively, the elasticity may be varied along or across base 102.

The degree of flexibility of base 102 is determined by the material of construction, the shape and dimensions of the device. Also, depending on the type of material used, the thickness of base 102 as well as its width and length may determine the flexibility of the device. The shape and dimensions of base 102 can be modified to change the flexibility of vaginal drug delivery device 100. In one embodiment, base 102 may comprise of a single material or combinations of different materials.

In one embodiment, base 102 may be pre-fabricated into different shapes. In one embodiment, base 102 has sharp corners. In one embodiment, base 102 has round corners.

Plurality of microstructures 104 each comprise a proximal end 106, a distal end 108, a body 110 and a tip 112. Microstructure 104 can be either straight or curved. In some embodiments, body 110 connects proximal end 106 to distal end 108 without curvature along its length. In one embodiment, body 110 is curved along its length between proximal end 106 to distal end 108. In one embodiment, microstructures 104 may have may shapes. In one embodiment, microstructures 104 may be canted or erect. In one embodiment, the general structure of microstructures 104 is of a rose thorn shape. In one embodiment, microstructures 104 are selected from the group consisting of microneedles, microblades, microanchors, microfishscale, micropillars, microhairs, and combinations thereof. Microstructures 104 can have a sharp tip 112 enabling it to penetrate into tissue, or can have a blunt tip 112 that enables it to merely grasp tissue without actual penetration. In one embodiment, microstructures 104 are designed to penetrate tissue to specific depths.

In one embodiment, microstructures 104 may have a circular cross-section or non-circular cross-section at proximal end 106. In one embodiment, microstructures 104 may have a cross-sectional diameter ranging between 80-600 μm at proximal end 106.

Vaginal drug delivery device 100 of the present invention may comprise microstructures 104 of any desired size, dimension, and geometry. Additionally, microstructures 104 may optionally comprise surfaces which are substantially smooth, or which comprise uneven surfaces, e.g., a microstructure comprising sides which are wavy, or which comprise protrusions, indentations, or depressions. For example, body 110 can have concave surfaces, convex surfaces, or a combination of concave and convex surfaces. In one embodiment, body 110 comprises at least one concave surface. In one embodiment, body 110 comprises at least one convex surface. In one embodiment, body 110 comprises at least one concave surface and at least one convex surface.

Tip 112 is located at distal end 108. In one embodiment, tip 112 can be selected from a group consisting of: a cube, a rectangle, a sphere, a cone, a pyramid, a cylinder, a tube, a ring, a tetrahedron, a hexagon, an octagon, or any irregular shapes. In one embodiment, the dimension (e.g., a diameter) of tip 112 may be within a range of about 10 nm to 1 μm.

The density, distribution, length, and orientation of microstructures 104 on base 102 may be modified depending on the type of application. Microstructures 104 may be bent or curve gradually, with distal end 108 directed at an optimal angle relative to base 102 to aid device penetration and stability within the tissue, and to reduce tissue irritation after installation. Microstructures 104 may be canted in one direction, such as toward the center of vaginal drug delivery device 100. Microstructures 104 may also be variously oriented, such as toward center and erect, or toward center and away from center. It is within the scope of this invention to have microstructures 104 extending in any relative direction or orientation on base 102.

In one embodiment, vaginal drug delivery device 100 of the present invention comprises microstructures 104 at an angle relative to base 102. Microstructures 104 may be positioned at any suitable angle. In one embodiment, microstructures 104 are affixed at an angle relative to base 102, wherein the angle is approximately 15, 30, 45, 60, 75, or 90 degrees, including all integers (e.g., 16°, 17°, 18°, etc.) and ranges (e.g., 15°-90°, 30°-90°, 45°-70°, etc.) in between of the angles set forth. In one embodiment, vaginal drug delivery device 100 of the present invention also include microstructures 104 with an angle relative to base 102, that is variable depending on its position in any microstructure array. In one embodiment, microstructures 104 may be angled in any direction. In some embodiments, all microstructures 104 in a particular array are angled in the same direction, or in approximately the same direction; while in other embodiments they are not.

In one embodiment, microstructures 104 of various lengths emanate from a single base 102. For example, in one embodiment, microstructures 104 are progressively shorter the closer they are to the center of vaginal drug delivery device 100. In one embodiment, microstructures 104 may also become progressively shorter the farther they are from the center of vaginal drug delivery device 100. In one embodiments, the length of an individual microstructure 104 may be ranging between 250-1000 μm. It may be desirable, in certain embodiments, to adjust the length of a microneedle according to the application/use and/or a payload delivered by vaginal drug delivery device 100.

The density of microstructures 104 may be predetermined and may vary depending upon the size of vaginal drug delivery device 100. In one embodiment, the density may be about or greater than about 100,000/cm², about 10,000/cm², about 5,000/cm², about 1,000/cm², about 500/cm², about 100/cm², about 50/cm², about 10/cm², or even about 1/cm².

Materials used for a microstructure 104 or a portion thereof may be selected and adapted for a particular use or design. Microstructures 104 can comprise a therapeutic agent. For instance, a therapeutic agent can be used in its crystallized or lyophilized state.

In one embodiment, microstructures 104 can comprise a degradable polymer. Without wishing to be bound by any particular theory, the degradable portion of microstructures 104 and the degradation rate may dictate the mechanism and efficiency of delivery of a therapeutic agent or other functions of vaginal drug delivery device 100. For instance, microstructure 104 can include or introduce a therapeutic agent so that the therapeutic agent is released after the degradation of microstructure 104. In one embodiment, base 102 comprises a degradable material. In certain embodiments, base 102 degrades so that microstructure 104 is released from vaginal drug delivery device 100 and may remain lodged in the internal tissue after interaction and/or implantation. In one embodiment, microstructure 104 may remain in the target tissue for several days. In one embodiment, microstructure 104 may remain in the target tissue for a duration of about 4-10 days. In one embodiment, microstructure 104 can remain in the target tissue for more than 10 days.

In certain embodiments, microstructure 104 lodged in the internal tissue may gradually degrade. In one embodiment, tip 112 comprises a degradable material. In one embodiment, tip 112 of a microstructure 104 degrades so that only tip 112 of the microstructure 104 breaks off. In one embodiment, microstructures 104 may be coated with a therapeutic agent. In one embodiment, base 102 may be coated with a therapeutic agent.

Suitable degradable polymers, and derivatives or combinations thereof, as discussed above can be selected and adapted to have a desired degradation rate. Alternatively, a degradation rate may be fine-tuned by associating or mixing other materials as previously described (e.g., non-degradable materials) with one or more of degradable polymers.

Vaginal drug delivery device 100 may comprise any material or mixture of materials. In one embodiment, vaginal drug delivery device 100 can comprise one or more biocompatible materials. Exemplary materials include, but are not limited to, metals (e.g., gold, silver, platinum, steel or other alloys); metal-coated materials; metal oxides; plastics; ceramics; silicon; glasses; mica; graphite; hydrogels; and polymers such as non-degradable or biodegradable polymers; and combinations thereof. Vaginal drug delivery device 100 may comprise one or more materials. In general, materials can be utilized in any form (e.g., lyophilized or crystallized) and/or for different purposes (e.g., therapeutics, diagnostics, etc.)

In some embodiments, vaginal drug delivery device 100 can comprise a magnetic material. For examples, a magnetic material can be utilized for positioning vaginal drug delivery device 100 in a target site or orientation, to trigger delivery of a therapeutic agent, or to affect interaction of the microstructure 104 to an internal tissue or a vessel wall.

In some embodiments, vaginal drug delivery device 100 can comprise deformable materials (e.g., polymers). As an example, vaginal drug delivery device 100 can comprise a deformable rubber so that the device swells enabling interaction of microstructure 104 protruding from base 102 to a tissue. In another example, a deformable vaginal drug delivery device 100 may be able to change size depending on pressure so that it can pass through lumens with diameters smaller than that of the device.

In some embodiments, vaginal drug delivery device 100 can comprise adhesive materials (e.g., adhesive polymers). An adhesive material may be used to bring vaginal drug delivery device 100 close to an internal tissue or a vessel wall facilitating the interaction of microstructures 104. Adhesiveness of vaginal drug delivery device 100 can aid in fixing/implanting at a target site for a prolonged period of time. In one embodiment, vaginal drug delivery device 100 may be treated with oxygen plasma to improve tissue adhesion properties.

In some embodiments, vaginal drug delivery device 100 can comprise one or more polymers. For example, a portion of vaginal drug delivery device 100 (e.g., microstructures 104) and/or a coating can comprise one or more polymers. Various polymers and methods known in the art can be used. Polymers may be natural polymers or unnatural (e.g. synthetic) polymers. In some embodiments, polymers can be linear or branched polymers. In some embodiments, polymers can be dendrimers. Polymers may be homopolymers or copolymers comprising two or more monomers. In terms of sequence, copolymers may be block copolymers, graft copolymers, random copolymers, blends, mixtures, and/or adducts of any of the foregoing and other polymers.

A polymer used in accordance with the present application can have a wide range of molecular weights. In some embodiments, the molecular weight of a polymer is greater than about 5 kDa. In some embodiments, the molecular weight of a polymer is greater than about 10 kDa. In some embodiments, the molecular weight of a polymer is greater than 50 kDa. In some embodiments, the molecular weight of a polymer is within a range of about 5 kDa to about 100 kDa.

In some embodiments, polymers may be synthetic polymers, including, but not limited to, polyethylenes, polycarbonates (e.g. poly(1,3-dioxan-2-one)), polyanhydrides (e.g. poly(sebacic anhydride)), polyhydroxyacids (e.g. poly(β-hydroxyalkanoate)), polypropylfumarates, polycaprolactones, polyamides (e.g. polycaprolactam), polyacetals, polyethers, polyesters (e.g. polylactide, polyglycolide), poly(orthoesters), polycyanoacrylates, polyvinyl alcohols, polyurethanes, polyphosphazenes, polyacrylates, polymethacrylates, polyureas, polystyrenes, and polyamines and copolymers thereof. In some embodiments, polymers include polymers which have been approved for use in humans by the U.S. Food and Drug Administration (FDA) under 21 C.F.R. § 177.2600, including, but not limited to, polyesters (e.g. polylactic acid, poly(lactic-co-glycolic acid), polycaprolactone, polyvalerolactone, poly(1,3-dioxan-2-one)); polyanhydrides (e.g. poly(sebacic anhydride)); polyethers (e.g., polyethylene glycol); polyurethanes; polymethacrylates; polyacrylates; polycyanoacrylates; copolymers of PEG and poly(ethylene oxide) (PEO).

In some embodiments, polymers used herein can be a degradable polymer. Such a degradable polymer can be hydrolytically degradable, biodegradable, thermally degradable, and/or photolytically degradable polyelectrolytes. For example, degradation of vaginal drug delivery device 100 comprising a degradable polymer can be induced by the ingestion of a solution targeted to specifically degrade vaginal drug delivery device 100 or a portion of the device (e.g., at least one microstructure 104).

Degradable polymers known in the art include, for example, certain polyesters, polyanhydrides, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Another exemplary degradable polymer is poly (beta-amino esters), which may be suitable for use in accordance with the present application. Another exemplary polymer is PCL that has a slow degradation and high permeability to many drugs while being nontoxic. The degradation of PCL can range from 4 weeks to 9 months by changing the polymer's molecular weight and the processing condition during the formation of the drug delivery device.

In one embodiment, vaginal drug delivery device 100 has a drug release rate of about 5-10 μg per day. In one embodiment, vaginal drug delivery device 100 has a drug release rate of more than 10 μg per day.

In one embodiment, for a shorter delivery times, poly(lactic acid) (PLA), poly(glycolic acid) (PGA) homopolymers, and poly(d,l-lactide-co-glycolide) (PLGA) copolymer can be used. These materials have a degradation rate ranging from 5 days to 3 months. PLGA undergoes hydrolysis in the body to produce the original monomers, lactic acid and glycolic acid. Since these two monomers are by-products of metabolic pathways in the body, there is minimal systemic toxicity associated with using PLGA for drug delivery or biomaterial applications.

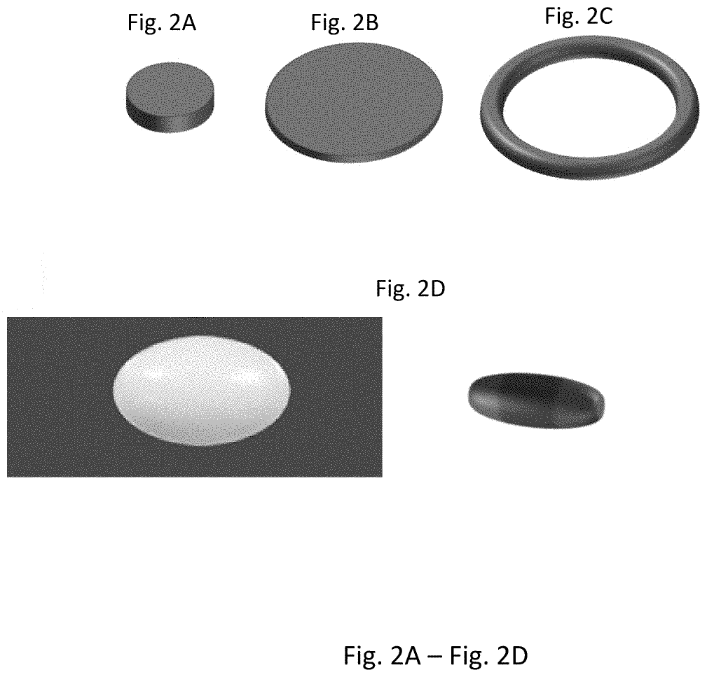

Referring now to FIG. 2, various exemplary forms of the vaginal drug delivery device 100 of the present invention is shown. In one embodiment, vaginal drug delivery device 100 can have any desired structure, including but not limited to tablets, films, rings, capsules and etc.

In one embodiment, vaginal drug delivery device 100 may be molded, stamped, machined, woven, bent, welded or otherwise fabricated to create the desired features and functional properties.

In one embodiment, vaginal drug delivery device 100 can be applied by the patient. In one embodiment, the vaginal drug delivery device can be applied by the physician.

### Therapeutic Agents

A therapeutic agent can be in a gas form, a liquid form, a solid form or combinations thereof. In some embodiments, the volume of a therapeutic agent may be in a range of about 0.1 mL to about 50 mL. In certain embodiments, a therapeutic agent of the disclosed vaginal drug delivery device 100 is carried in or transported through microstructures 104. An exemplary volume of a therapeutic agent carried within microstructures 104 can be within a range of about 1 nL to about 1 μL.

In accordance with the present disclosure, a therapeutic agent can include one or more agents for delivery after administration/implantation. A wide range of agents may be used. Agents may include, but are not limited to, therapeutic agents and/or an imaging agent. For example, agents may comprise any therapeutic agents (e.g. antibiotics, NSAIDs, angiogenesis inhibitors, neuroprotective agents, chemotherapeutic agents), cytotoxic agents, diagnostic agents (e.g. sensing agents, contrast agents; radionuclides; and fluorescent, luminescent, and magnetic moieties), prophylactic agents (e.g. vaccines), and/or nutraceutical agents (e.g. vitamins, minerals, etc.), or other substances that may be suitable for introduction to biological tissues, including pharmaceutical excipients and substances for cosmetics, and the like. In some embodiments, a therapeutic agent includes one or more bioactive agents.

An agent may comprise small molecules, large (i.e., macro-) molecules, any combinations thereof. Additionally or alternatively, an agent can be a formulation including various forms, such as liquids, liquid solutions, gels, hydrogels, solid particles (e.g., microparticles, nanoparticles), or combinations thereof.

In representative non-limiting embodiments, an agent can be selected from among amino acids, vaccines, antiviral agents, nucleic acids (e.g., siRNA, RNAi, and microRNA agents), gene delivery vectors, interleukin inhibitors, immunomodulators, neurotropic factors, neuroprotective agents, antineoplastic agents, chemotherapeutic agents, polysaccharides, anti-coagulants, antibiotics, analgesic agents, anesthetics, antihistamines, anti-inflammatory agents, vitamins and/or any combination thereof. In some embodiments, an agent may be selected from suitable proteins, peptides and fragments thereof, which can be naturally occurring, synthesized or recombinantly produced.

In some embodiments, an agent can comprise a cell. Such a device can be useful for the injection of whole cells (e.g., stem cells). In some embodiments, an agent comprises a biologic. Examples of biologics including, but are not limited to, monoclonal antibodies, single chain antibodies, aptamers, enzymes, growth factors, hormones, fusion proteins, cytokines, therapeutic enzymes, recombinant vaccines, blood factors, and anticoagulants. Exemplary biologics suitable for use in accordance with the present disclosure are discussed in S. Aggarwal, Nature Biotechnology, 28:11, 2010, the contents of which are incorporated by reference herein.

A therapeutic agent used in accordance with the present application can comprise an agent useful in combating inflammation and/or infection. A therapeutic agent may be an antibiotic. Exemplary antibiotics include, but are not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof. Other anti-microbial agents such as copper may also be used in accordance with the present invention. For example, anti-viral agents, anti-protazoal agents, anti-parasitic agents, etc. may be of use. Additionally or alternatively, a therapeutic agent may be an anti-inflammatory agent.

A therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. In another example, an antibiotic may be combined with an inhibitor of the enzyme commonly produced by bacteria to inactivate the antibiotic (e.g., penicillin and clavulanic acid).

In some embodiments, a therapeutic agent may be any therapeutic gene as known in the art. In some embodiments, a therapeutic agent is a non-viral vector. Typical non-viral gene delivery vectors comprise DNA (e.g., plasmid DNA produced in bacteria) or RNA. In certain embodiments, non-viral vectors are used in accordance with the present invention with the aid of a delivery vehicle. Delivery vehicles may be based around lipids (e.g., liposomes) which fuse with cell membranes releasing a nucleic acid into the cytoplasm of the cell. Additionally or alternatively, peptides or polymers may be used to form complexes (e.g., in form of particles) with a nucleic acid which may condense as well as protect the therapeutic activity as it attempts to reach a target destination.

Alternatively, a therapeutic agent can include one or more surfactants. Various surfactants are known in the art and can be suitable for use as an enhancer to increase tissue permeability for delivery.

A therapeutic agent used in accordance with the present application can comprise an agent useful in promoting cell migration and proliferation.

### Coatings

In accordance with the present disclosure, vaginal drug delivery device 100 can comprise a coating. In some embodiments, the surface of vaginal drug delivery device 100 may be coated. In some embodiments, a portion of vaginal drug delivery device 100 may be coated, such as one or more microstructures 104. In some embodiments, base 102 is coated. It will be appreciated that a coating may comprise one or more materials/units/layers.

In some embodiments, a coating comprises a payload, which may include one or more agents for delivery. A coating may be a medicated coating being made of or including an agent such as an anti-microbial agent. For example, an anti-microbial agent (e.g., gentamicin, clindamycin, copper, copper ions, silver) and/or a material with an ability to induce anti-microbial activity (e.g., gold that can be heated with an electromagnetic, magnetic, or electric signal) can be coated onto a device or a portion of a device. In another example, a coating can be utilized to carry a payload/agent. In certain embodiments, an agent can be associated with individual layers of a multilayer coating for incorporation, affording an opportunity for exquisite control of loading and release from the coating. For instance, an agent can be incorporated into a multilayer coating by serving as a layer.

In some embodiments, a coating comprises a targeting material such as antibodies, aptamers). Such coatings or materials can be used in combination with any other coating disclosed therein.

In some embodiments, a coating comprises an adhesive material as discussed above. For example, a coating can comprise a bioadhesive such as chitosan and carbopol. Such coatings or materials can be used in combination with any other coating disclosed therein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### Example 1: Vaginal Drug Delivery Device

This invention provides a technological platform to develop next-generation intravaginal drug delivery devices. Engineered devices are easy to make and scale up with bioresorbable and biocompatible properties. These devices are developed based on FDA-approved synthetic polymers like Polycaprolactone (PCL) and Poly Lactic-co-Glycolic Acid (PLGA). It is demonstrated that these devices can have prolonged and sustained release of hormone drugs (such as progesterone) for more than two months and sustained release of antibiotics (such Metronidazole and Clotrimazole) for 1-2 weeks period. The in vitro analysis has confirmed that the developed devices have no cytotoxicity characteristics. These devices were fabricated and tested in various shapes, including disk, film/sheet, ring, cone, and capsule, to optimize the effect of shape factor on the biological performance of the device. These drug delivery devices also provide the required structural flexibility to help their intravaginal insertion. The objective of this invention is to translate the basic insights gained in this study into important and relevant clinical applications to enhance women's health. This is a promising drug delivery approach for vaginal-related diseases. The engineered drug delivery platform of this invention addresses all the challenges preventing proper long-term vaginal treatment by offering inexpensive biodegradable materials with desirable long/short-term release profile of variety of therapeutics. The engineered device of the present invention can replace the current standard of care and enhance patient compliance due to its ease of use and competitive efficiency. It further integrates better with the vaginal mucosa by its tissue adhesiveness leading to expedited healing and more comfort. The vaginal drug delivery device of the present invention has several advantages: manufactured based on FDA-Approved/cGMP-grade bioresorbable polymers, platform to administer broad range of medicine (from antibiotics to hormones), tunable release profile ranging from 1 day to 5 months, completely bioresorbable (tunable biodegradation) which eliminate the use of removal, tissue adhesive (mucoadhesive) to expedited healing and improve the patient comfort, not messy like current creams on the market, easy to use for patients and easy to scale up in GMP facilities.

### Fabrication Process

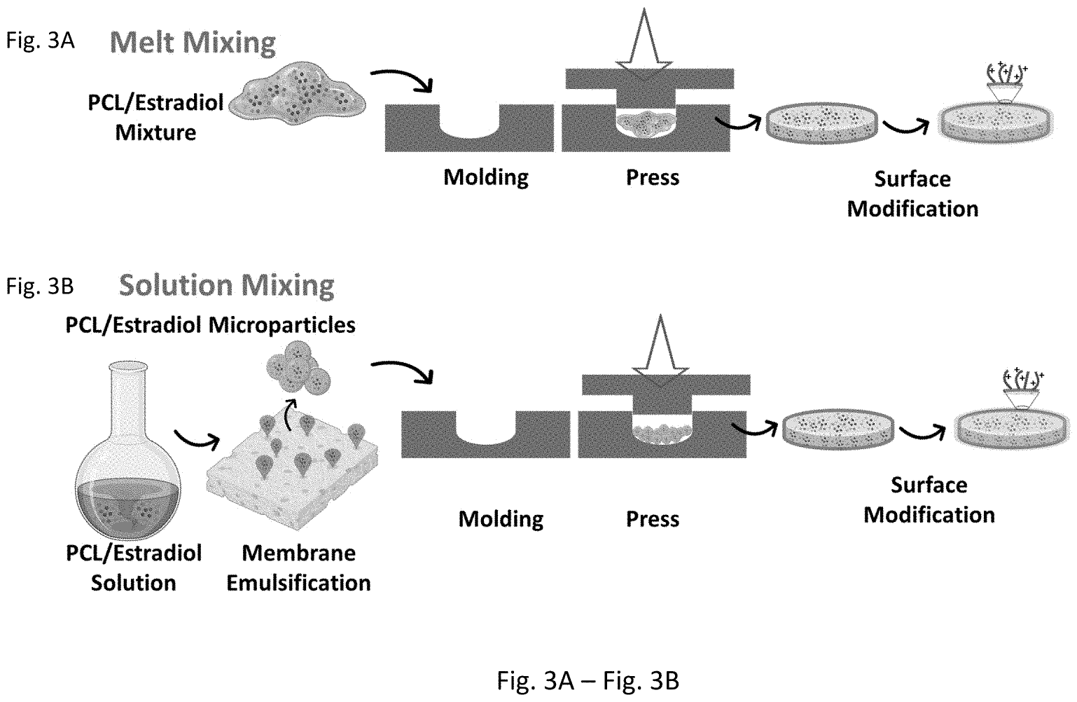

A series of drug delivery devices were developed based on FDA-approved synthetic polymers. cGMP-grade Polycaprolactone (PCL) and Poly Lactic-co-Glycolic Acid (PLGA) were used as based polymers due to their well-studied biocompatibility and their wide use in FDA-approved medical devices. Two techniques were used to develop the vaginal drug delivery devices of the present invention (FIG. 3A, FIG. 3B). First, melt processing was utilized. This process can be performed in lab or industrial scale compounders or extruders and the device can be made by casting and hot pressing in the laboratory scale or using injection molding process for industrial scales. Melt processing has a benefit of being more scalable with less environmental concern as the process benefits from solvent-free approach. A solvent-based process was used to first form polymer/drug microparticles using either homogenizing or membrane emulsification methods. Membrane emulsification is a scalable process that can form extremely monodisperse particles. Here the solution of polymer-drug mixture was prepared in dichloromethane (DCM) and then passed through micron-size filters into non-solvent (here water) media. The pore size and processing conditions (e.g., polymer concentration, temperature, infusion rate, and mixing speed) can control the size of polymer/dug microparticles.

To improve the tissue adhesion properties, devices were treated with oxygen plasma for 2-5 min (each side) and then immersed in 1 M Sodium Hydroxide (NaOH) and placed on gentle shaking for 4 h at room temperature. The treated devices were washed three times in deionized water and dried using nitrogen gas flow. Following the surface activation, the surface conjugated with either chitosan (medium molecular weight chitosan with molecular weight of 280,000 g/mol and degree of deacetylation of 83%) or Poly-L-lysine (PLL). First, devices were immersed in crosslinking solution containing 1-Ethyl-3-(3-dimethylaminopropyl)carbodiimide (EDC; 200 mg·mL⁻¹) and N-hydroxysuccinimide (NETS; 50 mg·mL⁻¹) in 2-(N-morpholino) ethane sulfonic acid (MES, pH 5.9) for 10 minutes and then washed with PBS. After the addition of either chitosan or PLL polymers, the devices were left under gentle shaking overnight at 4° C. Then the devices were washed with NaCl (0.15 M; pH 8) for 30 min.

FDA-approved tablet form of estradiol (Vagifem®) as well as vaginally administered rings (Femring®) were used as templates for designing the shape. Here tablet, film, ring, and tablet forms were fabricated and tested to optimize the design in term of flexibility, release, and degradation rates (FIG. 2A, FIG. 2B).

The primary investigation was performed on PCL, which is an aliphatic poly(α-hydroxy acid) and semi-crystalline polymer. The degradation of PCL depends on chemical hydrolysis of hydrolytically labile aliphatic ester linkages. Owing to its slow degradation, high permeability to many drugs and nontoxicity, PCL was initially investigated as a long-term drug delivery vehicle, for example, the long-term contraceptive device Capronor. This biodegradable PCL capsule device was implanted sub-dermally and was capable of long term zero-order controlled release of levonorgestrel. PCL alone is stiff and has a slow degradation profile. The degradation half-life of PCL can be tuned between 4 weeks to several months by changing the polymer's molecular weight and the processing condition during the formation of the drug delivery device as it will affect the polymer crystallinity.

### Hormone Delivery

Release and Bioactivity of Estradiol

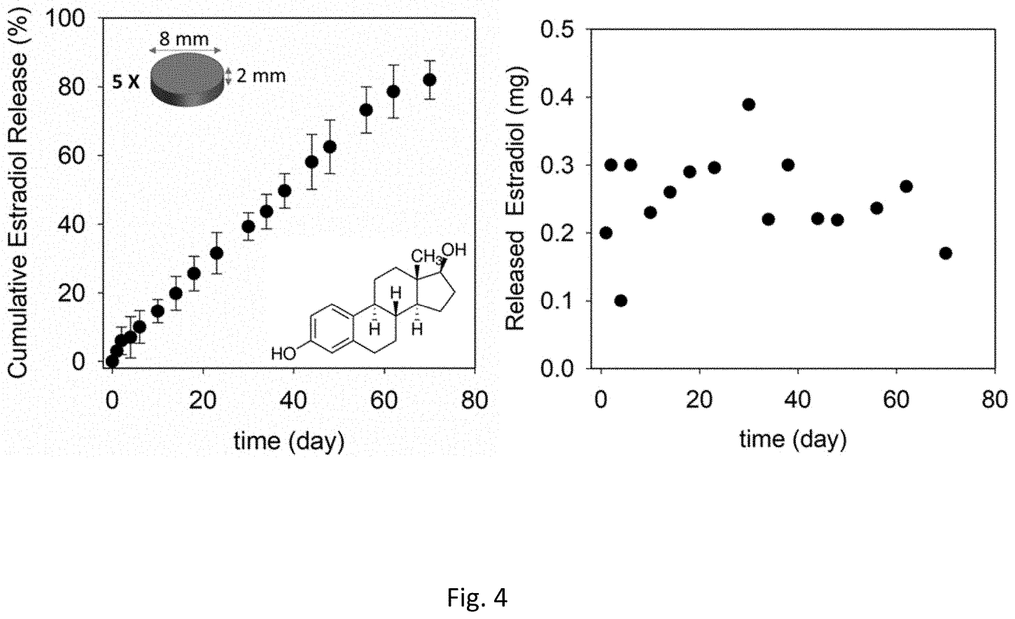

Estradiol and progesterone were used as model drugs to fabricate vaginal drug delivery devices. The loading dosages were varied from 20 ug to 40 mg per device. The approximate release rate is 5-20 ug per device per day. The total amount and the release rate can be controlled by altering the design as well as the encapsulated content to match the desired therapeutic regime. As example cumulative release of Estradiol was shown in FIG. 4. In vitro release of estradiol was studied by incubating five devices in PBS (pH 7.4) at 37° C. At different time intervals, 800 μL of the supernatant was separated from samples and replaced with an equivalent volume of fresh PBS solution. The concentration of released gentamicin was determined by measuring the UV absorption.

### Bioactivity of Released Estradiol

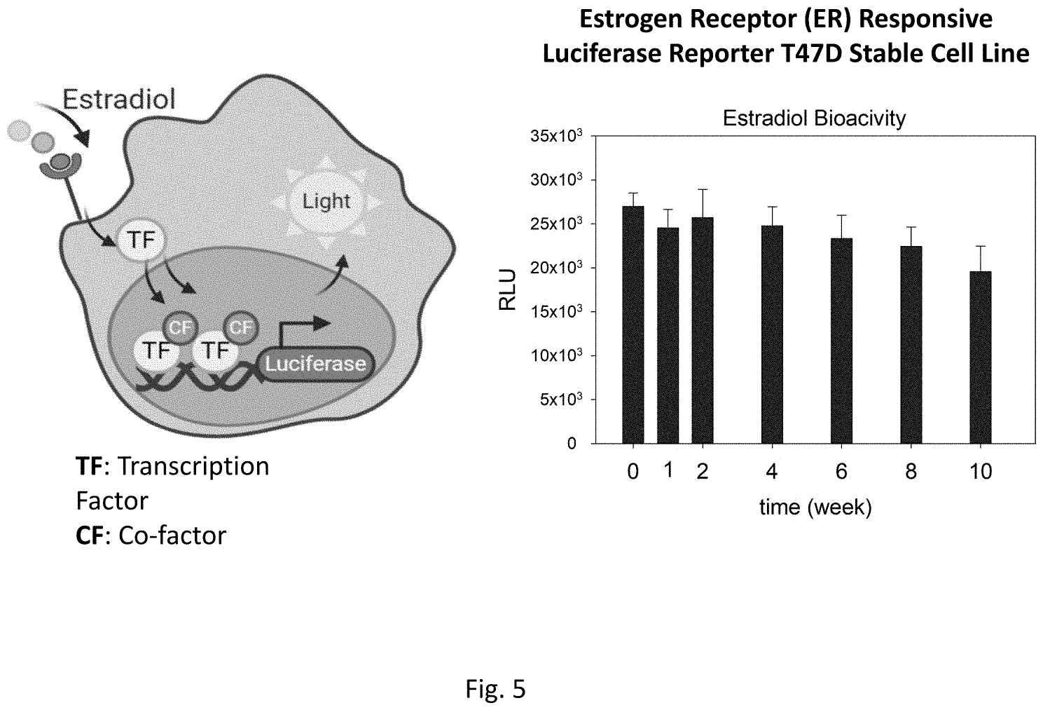

In order to check the stability and bioactivity of release estradiol from engineered drug delivery devices, estrogen Receptor (ER) Responsive Luciferase Reporter T47D Stable Cell Line was used. This cell line is derived from human breast cancer, and stably express firefly luciferase reporter gene under the control of the ER response element. This cell line is known as ideal cellular model for monitoring the activation of Estrogen Receptor Signaling Pathway triggered by stimuli treatment. The activity of release estradiol from PCL device were monitored weekly using the mentioned cell reported cell line (FIG. 5). Results demonstrate release drugs retain their bioactivity as they can successfully bind to estrogen Receptor and trigger the underlying cellular pathway.

### Hormone Delivery

Release and Bioactivity of Progesterone

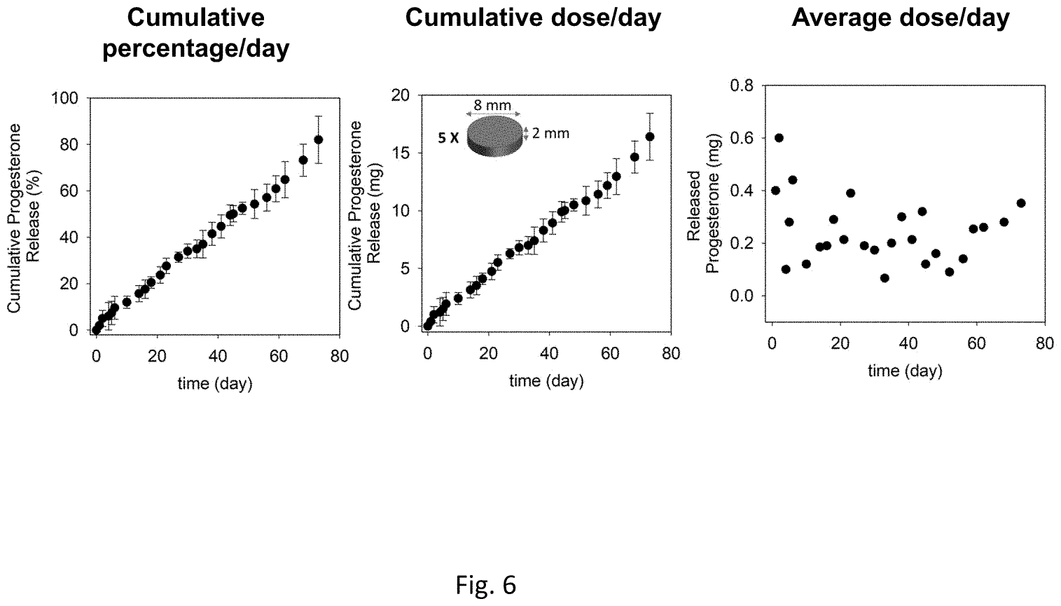

PCL devices loaded with progesterone shows the similar release profile as shown in FIG. 6. These results demonstrate the universality of this platform for prolonged release of target hydrophobic vaginal drugs.

PLGA for Fast and PCL for Sustained Release

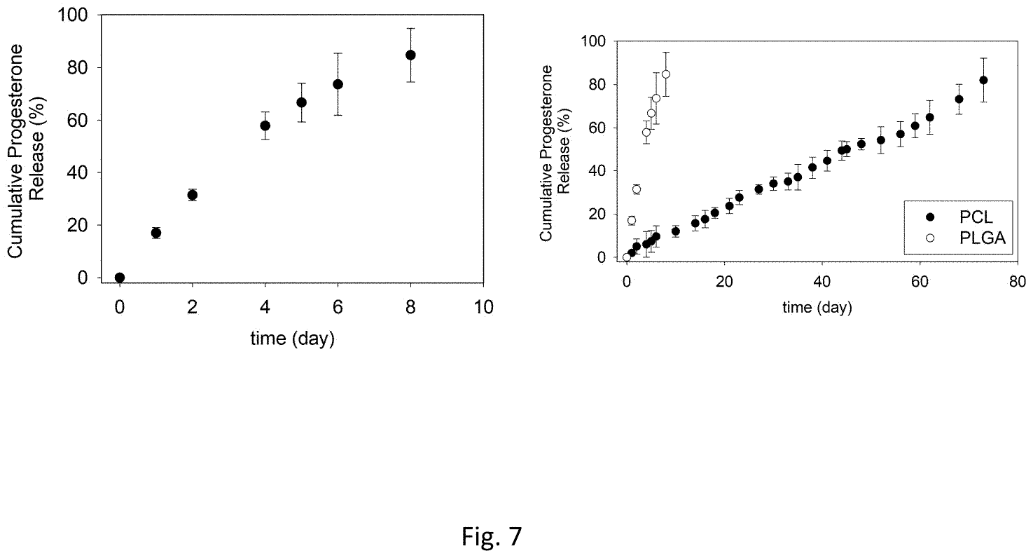

For shorter delivery period, poly(lactic acid) (PLA), poly(glycolic acid) (PGA) homopolymers, and poly(d,l-lactide-co-glycolide) (PLGA) copolymer was used. The PLA homopolymer is stiff due to its highly crystalline nature, while PGA homopolymer is soft due to low crystallinity. Depending on the ratio of lactide to glycolide used for polymerization, different forms with broad range of degradation periods (5 days to 3 months) can be obtained. The higher the content of lactide units, the higher the molecular weight and crystalline content, and this results in slower degradation. PLGA undergoes hydrolysis in the body to produce the original monomers, lactic acid and glycolic acid. Since these two monomers are by-products of metabolic pathways in the body, there is minimal systemic toxicity associated with using PLGA for drug delivery or biomaterial applications. Here PLGA (50:50) was used to load and release progesterone overtime. As shown in FIG. 7, such a device can release its cargo in less than 10 days compared to more than 70 days for PCL-based devices.

### Antibacterial Delivery

PLGA for Hydrophobic and Chitosan or Laponite for Hydrophilic Drug Release

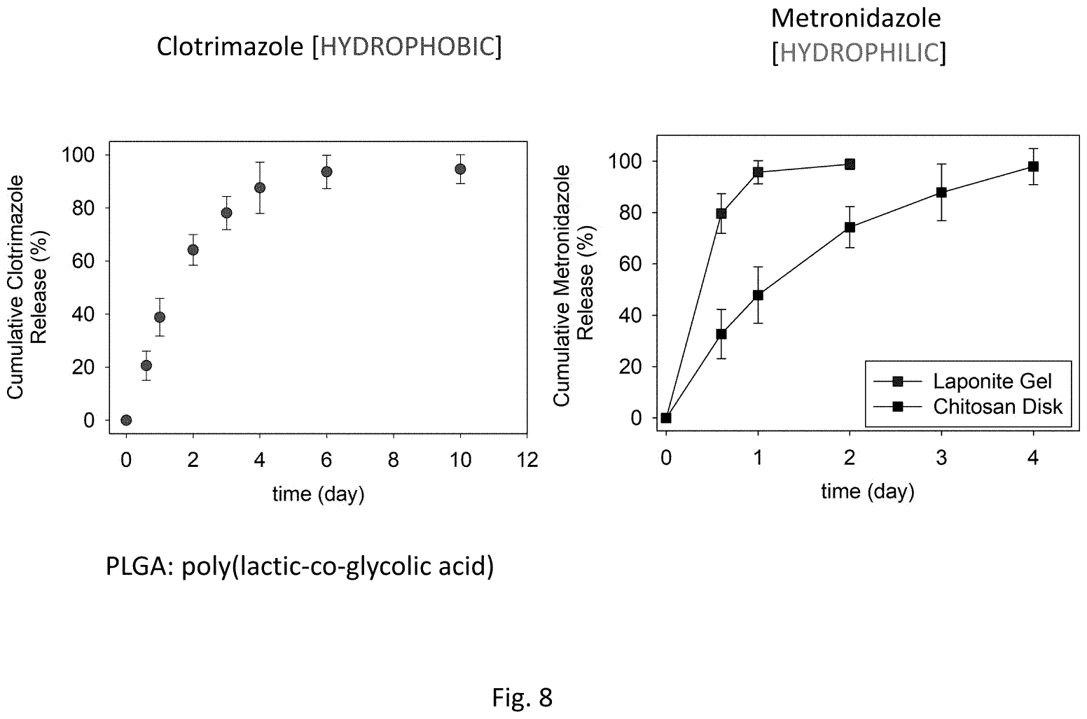

PLGA device and its fast release properties, make such device a good choice for antibacterial and antifungal hydrophobic drugs. Here this platform was utilized to load and release Clotrimazole which is an antifungal medication. It is used to treat vaginal yeast infections, oral thrush, diaper rash, Pityriasis versicolor, and types of ringworm including athlete's foot and jock itch. Sustained release up to 10 days make this device a good option to deliver this medication to treat vaginal infection (FIG. 8). This device increases therapeutic outcome and patient comfort compared to messy virginal creams by bringing new features like being easier to place and continuously release its medication for a week.

The device can also benefit from other types of polymer to load and deliver hydrophilic drugs like Metronidazole which is being used to treat vaginal infections. Here the use of Chitosan (crosslinked with glutaraldehyde) and laponite gel were tested as shown in FIG. 8.

### Antibacterial Properties

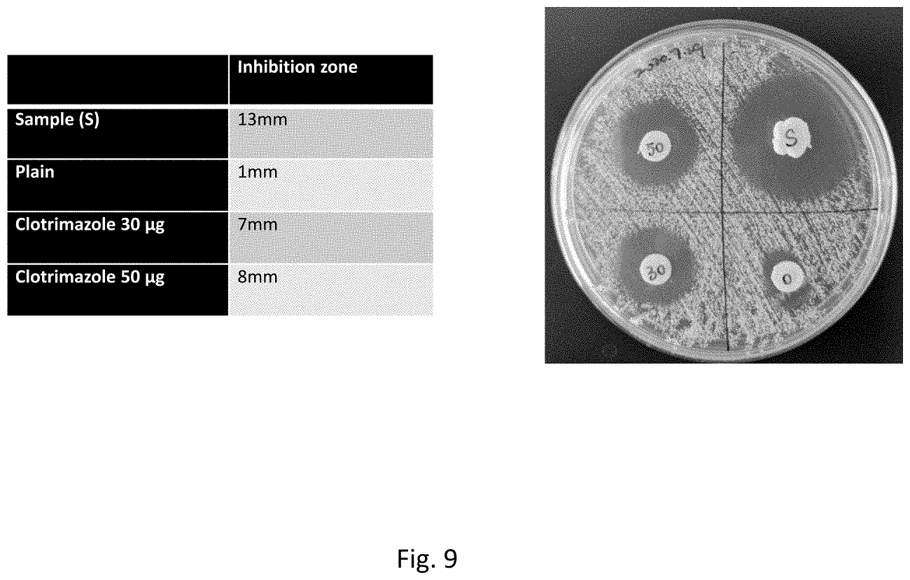

Two techniques were used to quantify the antibacterial properties of these device against an example vaginal yeast (Albicans). First, using disk diffusion assay. Here Candida albicans (ATCC 18804) was used. The yeast was cultured in Sabouraud dextrose broth and a thin layer was streaked evenly onto Sabouraud's agar using a sterilized cotton swab. Experimental sample, which contains 50 μg of clotrimazole in PLGA device, along with negative and positive paper disk controls were place onto the agar plate. After 24 h culture at 30° C. inhibition zone was examined for each condition, as shown in FIG. 9.

Liquid assay was also used to further characterize the antibacterial properties. Candida albicans (ATCC 18804) was cultured in Sabouraud dextrose broth with a starting concentration of 0.08 OD600 value. After 12 h, optical density was measured using spectrometer at 600 nm wavelength. Then, conversion to cell density is using the approximation 0.5 equals 1.5×10⁸ CFU/ml.

Clotrimazole-loaded PLGA disk has shown effective antibacterial properties towards the yeast stain Candida albicans, and a significant inhibition zone of 13 mm was observed in disk diffusion assay (FIG. 9). Also, antibacterial property of clotrimazole is observed in liquid assay with an IC50 (half maximal inhibitory concentration) of 3.3 μg/ml (Table 1).

TABLE 1
Inhibition of yeast growth in
Sabouraud dextrose broth.

| Clotrimazole | Optical | Cell Density |
|---|---|---|
| Conc. (μg/ml) | Density (OD600) | (×10⁸/ml) |
| 0 | 1.4 | 4.2 |
| 0.37 | 0.9 | 2.7 |
| 1.1 | 0.8 | 2.4 |
| 3.3 | 0.7 | 2.1 |
| 10 | 0.5 | 1.5 |

### In Vivo Biocompatibility of Delivery Devices

GMP-grade PLGA and PCL polymers were used. X-ray irradiation (Gulmay Medical RS320 x-ray unit) was also utilized to irradiate the fabricated device before in vitro or in vivo functional assays, following ISO 11137-2:2013 recommended protocols. A 25 kGy (2.5 Mrads) sterilization dose was used, since this dose does not alter drug properties. Physical properties, including changes in morphology and release rate of the devices as well as change in drug activity or antibacterial property change after sterilization, were tested. The results show non-significant changes in PLGA and PCL devices after receiving three cycles of 25 kGy.

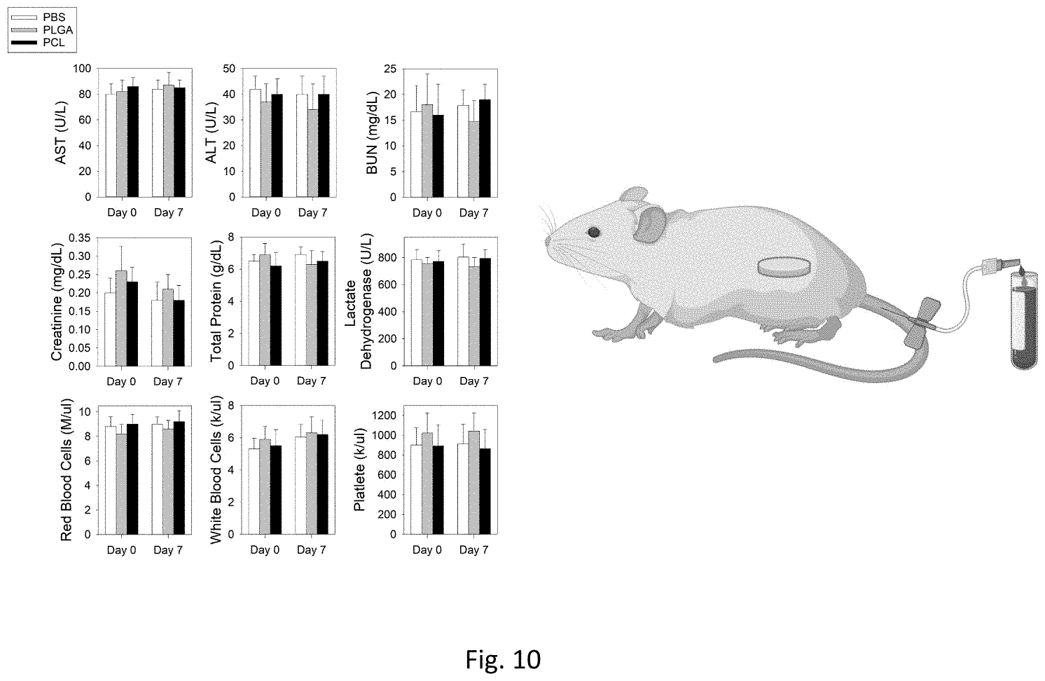

The in vivo biocompatibility of the engineered drug delivery devices were tested in wild-type mice using a subcutaneous implantation model. No signs of lymphocyte (CD3) or macrophage (CD68) infiltration were observed 7 days after subcutaneous implantation. Based on these results, no significant foreign body response to the PCL and PLGA devices is expected. Comprehensive testing was also performed on the blood of the recipient mice to check for any potential systematic toxicity of the devices. Comparing results to those of mice that went undergo surgical procedures but didn't receive any PCL or PLGA devices (Control: phosphate buffer saline (PBS)), there were no significant differences (p>0.05) in blood cells, including red blood cells, white blood cells, and platelets (FIG. 10). Metabolic screening 7 days after administration of various devices also showed no significant changes (p>0.05) in liver/kidney functions (FIG. 10).

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A vaginal drug delivery device comprising:
a flexible base; and
a plurality of microstructures protruding from the base, wherein the microstructures each comprise a proximal end, a distal end, a body and a tip.

2. The vaginal drug delivery device of claim 1, wherein the microstructures are selected from the group consisting of microneedles, microblades, microanchors, microfishscale, micropillars, microhairs and combinations thereof.

3. The vaginal drug delivery device of claim 1, wherein the microstructures each comprises a length ranging from about 250-1000 μm.

4. The vaginal drug delivery device of claim 1, wherein the microstructures each comprises a cross sectional diameter ranging from about 80-600 μm at the proximal end.

5. The vaginal drug delivery device of claim 1, wherein the base is biodegradable.

6. The vaginal drug delivery device of claim 1, wherein the plurality of microstructures are biodegradable.

7. The vaginal drug delivery device of claim 1, wherein the base has a shape selected from the group consisting of: a tablet, a film, a ring, a capsule, and combinations thereof.

8. The vaginal drug delivery device of claim 1, wherein the device is made from polymers selected from the group consisting of: Polyesters comprising Poly(glycolic acid), Poly(lactic acid), Poly(lactic-glycolic acid) and Poly(caprolactone) (PCL); Polysaccharides comprising chitosan, dextran, alginate, hyaluronic acid; Polyanhydrides; Polyorthoesters; Polyurethanes and combinations thereof.

9. The vaginal drug delivery device of claim 1, wherein the plurality of microstructures further comprise a therapeutic agent.

10. The vaginal drug delivery device of claim 10, wherein the therapeutic agent is a drug.

11. The vaginal drug delivery device of claim 11, wherein the drug is selected from the group consisting of: estrogen, progesterone, estradiol, an antibacterial agent, an antifungal agent, and combinations thereof.

12. The vaginal drug delivery device of claim 1, wherein the device has a degradation rate of about 4 days to 9 months after insertion.

13. The vaginal drug delivery device of claim 1, wherein the device has a drug release rate of about 5-20 μg per day.

14. The vaginal drug delivery device of claim 1, wherein the body is curved along its length between the proximal end and the distal end.

15. The vaginal drug delivery device of claim 1, wherein the body connects the proximal end and the distal end without any curvature along its length.

16. The vaginal drug delivery device of claim 1, wherein the tip is selected from the group consisting of: a cube, a rectangle, a sphere, a cone, a pyramid, a cylinder, a tube, a ring, a tetrahedron, a hexagon, an octagon, or any irregular shapes.
